(19) United States
(12) Patent Application Publication
(10) Pub. No.: US 2013/0114798 A1
(43) Pub. Date: May 9, 2013

(54) DIGITAL X-RAY FIELD AND LIGHT FIELD ALIGNMENT

(71) Applicants: David Eric Hintenlang, Archer, FL (US); Matthew Robert Hoerner, Gainesville, FL (US)

(72) Inventors: David Eric Hintenlang, Archer, FL (US); Matthew Robert Hoerner, Gainesville, FL (US)

(21) Appl. No.: 13/809,675

(22) PCT Filed: Jul. 14, 2011
(86) PCT No.: PCT/US11/44049

§ 371 Date: Jan. 11, 2013

Related U.S. Application Data
(60) Provisional application No. 61/365,295, filed on Jul. 16, 2010.

(51) Int. Cl. A61B 6/00

(52) U.S. Cl. 378/205

(57) ABSTRACT

Various methods and systems are provided for x-ray field alignment. In one embodiment, a system includes a plurality of scintillation detectors distributed about edges of a light field associated with an x-ray field, a detector interface in communication with the plurality of scintillation detectors, and a computing device in communication with the detector interface. The detector interface configured to simultaneously obtain exposure data for the plurality of scintillation detectors and the computing device configured to determine an alignment distance between the light field and the x-ray field based at least in part upon the exposure data. In another embodiment, a method includes obtaining exposure data from a plurality of scintillation detectors distributed about edges of a light field during irradiation by an x-ray field and determining an alignment distance between the light field and the x-ray field based at least in part upon the exposure data.

### CROSS REFERENCE TO RELATED APPLICATIONS

This application claims priority to copending U.S. provisional application entitled “DIGITAL X-RAY FIELD AND LIGHT FIELD ALIGNMENT” having Ser. No. 61/365,295, filed Jul. 16, 2010, which is entirely incorporated herein by reference.

### BACKGROUND

X-ray imaging is used in many diagnostic tests such as, e.g., mammography. Light fields are used to indicate the area irradiated by the x-ray field. In general, the center and edges of co-incident x-ray and light fields should coincide. Quality assurance often requires x-ray/light field sizes to be tested and maintained within alignment tolerance levels.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is an example of a system for digital x-ray field and light field alignment in accordance with various embodiments of the present disclosure.

FIGS. 2A and 2B are graphical representations of examples of a water-equivalent fiber optic coupled (FOC) dosimeter used in the system of FIG. 1 in accordance with various embodiments of the present disclosure.

FIG. 3 is a picture of a FOC dosimeter of FIG. 2B in accordance with various embodiments of the present disclosure.

FIG. 4 is a graphical representation of an example of a detector interface of FIG. 1 in accordance with various embodiments of the present disclosure.

FIG. 5 is an example of a graph of integrated counts versus exposed scintillator length (cm) for FOC dosimeter of FIGS. 2A and 2B in accordance with various embodiments of the present disclosure.

FIG. 6 is a flow chart illustrating an example of field alignment using the system of FIG. 1 in accordance with various embodiments of the present disclosure.

FIG. 7 illustrates an example of a screen for display by the computing system of FIGS. 1 and 4 in accordance with various embodiments of the present disclosure.

FIG. 8 illustrates an example of realtime data collected with the system of FIG. 1 in accordance with various embodiments of the present disclosure.

FIG. 9 is an example of the energy dependence of a FOC dosimeter of FIGS. 2A and 2B in accordance with various embodiments of the present disclosure.

FIG. 10 is an example of the change in the x-ray energy spectrum of a FOC dosimeter of FIGS. 2A and 2B with respect to tissue depth in accordance with various embodiments of the present disclosure.

### DETAILED DESCRIPTION

Disclosed herein are various embodiments of systems and methods related to digital x-ray field and light field alignment. Reference will now be made in detail to the description of the embodiments as illustrated in the drawings, wherein like reference numbers indicate like parts throughout the several views.

A light field is used to illuminate the area irradiated by a co-incident x-ray field. The alignment of the x-ray field with the light field is maintained to protect from improper exposure. The most conventional method of quantifying the x-ray/light field misalignment is using x-ray sensitive film. However, most facilities today have completely converted to digital x-ray imaging systems and do not have film processing capabilities. Therefore physicists must rely on radiochromatic film which has poor x-ray sensitivity resulting in a demonstrated lack of reproducibility and accuracy. Despite its wide practice, this process of quantifying the x-ray/light field misalignment using radiochromatic film is quite tedious due the placement of four different film dosimeters on each side of the light field. To darken the film, multiple exposures must be taken, which also makes the process more time consuming.

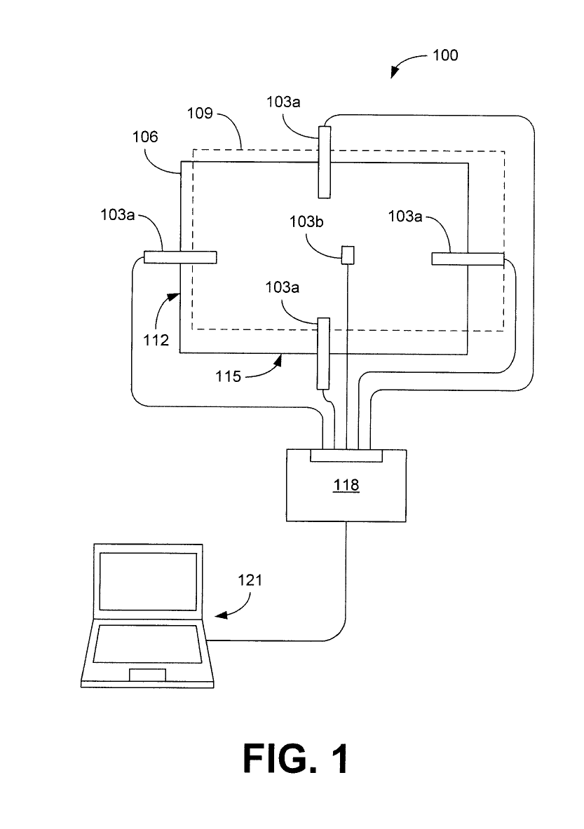

Referring to FIG. 1, shown is an example of a system 100 for digital x-ray field and light field alignment. The system 100 includes an array of scintillation detectors 103 that quantify the alignment between the light field 106 and x-ray field 109. One or more scintillation detectors 103a may be aligned optically with the edge of the light field 106, and register counts during each exposure. The total number of counts from a scintillation detector 103a correlates to a distance between the two fields 106 and 109. In addition, a scintillation detector 103b may be positioned within the center portion of the light field 106. For example, the scintillation detector 103b may be substantially centered within the light field 106. Hence, all sides of the x-ray field 109 along with the x-ray beam intensity may be evaluated simultaneously. The system 100 disclosed herein permits rapid and accurate measurements of the x-ray/light field alignment for a radiographic system such as, but not limited to, a mammography imaging system. By determining the field misalignment using x-rays, this method of quality assurance (QA) can be used on many types of radiographic systems. For example, QA protocol in mammography requires all field sizes and multiple lateral positions to be tested, which depending on the imaging system, can range between four and eight fields.

The scintillation detectors 103a aligned with the edge of the light field 106 may be substantially perpendicular to the edge of the light field 106. In the example of FIG. 1, the light field 106 and x-ray fields are rectangular. A first scintillation detector 103a may be aligned to be substantially perpendicular to a first edge 112 of the light field 106. A second scintillation detector 103a may be aligned to be substantially perpendicular to a second edge 115 of the light field 106, and thus, substantially perpendicular to the first scintillation detector 103a. The scintillation detectors 103a may be centered on the edge of the light field 106 to allow for variation in the x-ray field 109 in either direction.

The signals from the scintillation detectors 103 are directed to a detector interface 118 for preprocessing and delivery to a computing device 121 for further processing and/or evaluation. The system 100 permits accurate and real-time measurements of x-ray radiation fields 109 with continuous monitoring with time intervals as short as 10 msec. In some implementations, the evaluation results are rendered for display on a display device (e.g., the computing device display). In other implementations, the evaluation results may be utilized to control the position and/or focus of the x-ray field 109. For example, the positioning and/or alignment of a source of the x-ray field 109 may be adjusted based upon the evaluation results. Control signals and/or control data may be provided by the computing device 121 to adjust the x-ray source.

The scintillation detectors 103 may be, e.g., a water-equivalent fiber optic coupled (FOC) dosimeter. The FOC architecture provides the ability to obtain realtime dose information during irradiation of the device. It also offers a small device size for use in a phantom. Other dosimeters commonly used in diagnostic radiology, such as ionization chambers, thermoluminescent dosimeters (TLDs), or optically stimulated luminescent (OSL) dosimeters are either too large to incorporate into phantoms or require a time consuming reading process after irradiation to extract dose information. FOC dosimeters overcome many of the shortfalls of other dosimeter systems by showing little angular dependence, no detectable performance degradation over time, high reproducibility, and real-time output while maintaining a small physical size that allows measurements with high spatial resolution.

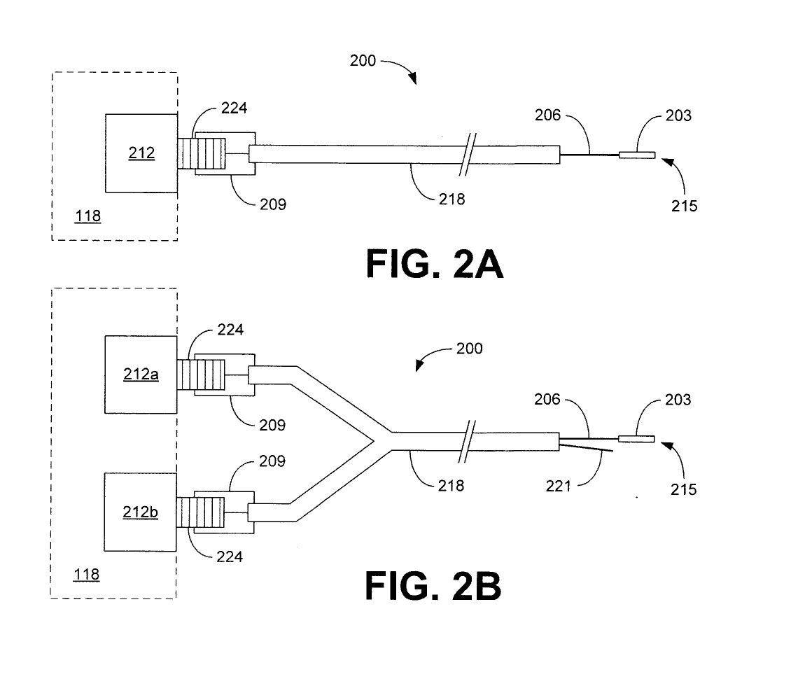

Referring to FIGS. 2A and 2B, shown are graphical representations of examples of a water-equivalent FOC dosimeter 200. In one embodiment, the FOC dosimeter 200 includes a plastic scintillator 203 coupled to an optical fiber 206. Plastic scintillation material is used as the sensitive element of the FOC dosimeter 200 to provide adequate light output to maintain a high signal-to-noise ratio and a water-equivalent effective Z. The water-equivalent effective Z of the plastic scintillator 203 prevents image artifacts and mimics the radiation interaction properties of soft tissue, eliminating an over-response to low energy photons and producing an energy dependence that allows for easy and effective calibration. The water equivalent effective atomic number of the plastic scintillator may provide advantages for human radiation dose applications as the atomic number is equivalent to that of human soft tissue.

In the embodiments of FIG. 2A, the FOC dosimeter 200 was constructed by coupling a small cylindrical plastic scintillator 203 to the optical fiber 206. The optical fiber 206 acts as a light guide to direct scintillation photons from the plastic scintillator 203 to the detector interface 118. The plastic scintillator 203 and the optical fiber 206 may be provided in a variety of diameters and lengths. The diameter of the plastic scintillator 203 may range from about 100 μm to about 1 mm and the length may range from about 1 mm to about 10 cm or more (possibly as large as the linear dimensions of the x-ray field 109). For example, the length of the plastic scintillator 203 may be in the range of about 2 cm to about 5 cm. The diameter of the optical fiber 206 may range from may range from about 100 μm to about 1 mm and the length of about 250 cm or more. The diameter of the plastic scintillator 203 may be selected to be about the same as the diameter of the optical fiber 206. In some embodiments, the plastic scintillator 203 (e.g., BCF-12, Saint-Gobain Crystals, Nemours, France) may be about 500 μm in diameter and about 2 mm in length and the optical fiber 206 (e.g., 400-UV, Ocean Optics Inc., Dunedin, Fla.) may be about 2 m in length and about 400 μm in diameter. Other diameters and lengths may be utilized as appropriate.

When stimulated by an x-ray radiation field 109 (FIG. 1), the plastic scintillator 203 generates scintillation photons. To transmit the scintillation photons from the plastic scintillator 203 to the detector interface 118, the scintillator 203 is coupled to one end of the optical fiber 206. A connector 209 (e.g., a female SMA 905 connector) may be coupled to the other end of the optical fiber 209 to enable the FOC dosimeter 200 to be connected to a readout device 212 included in the detector interface 118. To maximize the number of scintillation photons reaching the detector interface 118, the coupled ends of both the scintillator 203 and the optical fiber 206 may be polished. For example, the ends may be polished with progressively finer lapping films (e.g., 12, 3, and 1 μm). The uncoupled end 215 of the scintillator 203 may be similarly polished and coated with a reflective coating (e.g., a reflective paint) in order to prevent the escape of scintillation photons, effectively increasing the output of the FOC dosimeter 200.

In one implementation, the polished ends of the scintillator 203 and the optical fiber 206 may be mechanically coupled using a short piece (e.g., about 1 cm) of heat shrink tubing. In another implementation, the polished ends of the scintillator 203 and the optical fiber 206 are optically coupled using ethyl cyanoacrylate glue. The utilization of the glue as an optical coupler offers a more robust design than a simple mechanical coupling. In other implementations, combinations of optical gels and mechanical coupling may be used to provide optical coupling between the scintillator 203 and the optical fiber 206. The FOC dosimeter 200 may be then be coated to restrict ambient light and add strength to the assembly. For example, the FOC dosimeter 200 may be wrapped in opaque heat shrink 218.

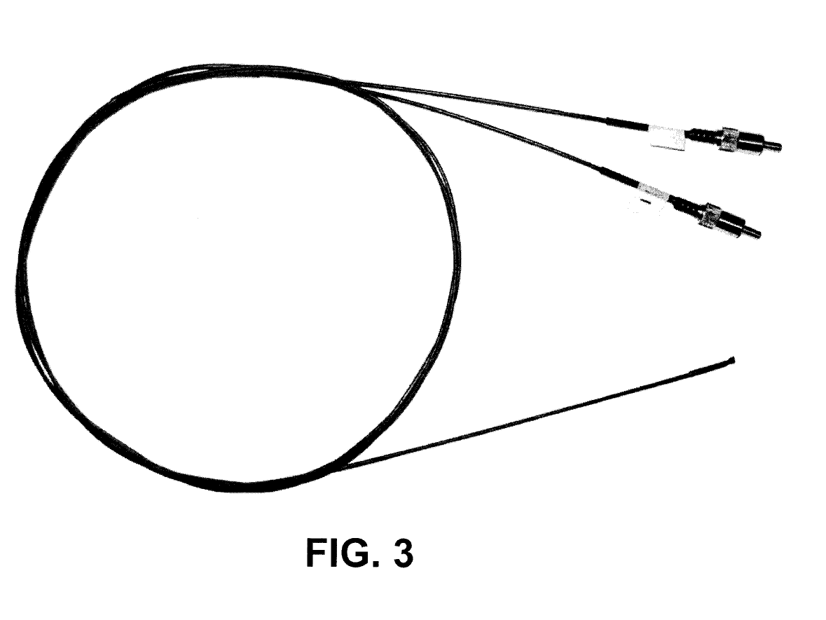

In the embodiment of FIG. 2B, a second optical fiber, known as a reference fiber 221, may also be included in the FOC dosimeter 200 to account for the native fluorescence of the optical fiber 206 itself, otherwise known as the background signal. The reference fiber 221 may be integrated into the FOC dosimeter 200 using a single piece of heat shrink tubing 218, which can be bifurcated at its end to allow the optical fiber 206 and the reference fiber 221 to be connected with its respective readout device 212a and 212b. The integration of the reference fiber 221 into the FOC dosimeter 200 provides for more accurate background subtraction than a separate reference fiber placed in the radiation field. Ultimately, this provides a user with a more accurate estimate of the radiation dose. Coupling both the signal fiber 206 and the reference fiber 221 may be achieved by providing, e.g., a female SMA (SubMiniature A) connector 209 to each fiber 206/221 and a male SMA connector 224 on each readout device 212. FIG. 3 is a picture of a FOC dosimeter including a reference fiber and bifurcated connections.

While the majority of the light reaching a readout device 212 through the optical fiber 206 is a result of photons released from the scintillator 203 of the FOC dosimeter 200, a fraction of the light is a result of the native fluorescence within the optical fiber 206 itself. The fluorescence of the optical fiber 206 is commonly referred to as the stem effect and if not accounted for can result in significant errors in dose measurements. In order to account for this effect, the reference fiber 221 was constructed without a scintillator. The length of the reference fiber 221 is about the same as the optical fiber 206. With the reference fiber 221 adjacent to the optical fiber 206, the stem effect exhibited by the reference fiber 221 during irradiation can be measured and subtracted from the measurement from the optical fiber 206 and plastic scintillator 203 to compensate for the stem effect of the optical fiber 206. This would normally be implemented for scintillation detectors 103 such as, e.g., FOC dosimeters 200 where a portion of the optical fiber 206 is in the x-ray field 109.

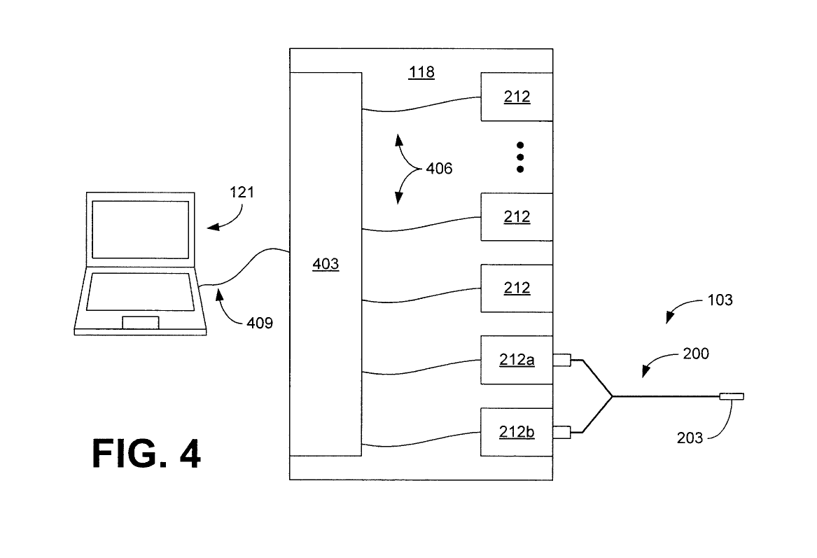

Referring next to FIG. 4, shown is a graphical representation of an example of the detector interface 118 of FIG. 1 in accordance with various embodiments of the present disclosure. The detector interface 118 includes one or more readout device(s) 212 such as, but not limited to a photomultiplier tube (PMT) and/or a multi-pixel photon counter (MPPC). A PMT takes the light signal from a scintillation detector 103 (e.g., a FOC dosimeter 200) and converts the light signal into an electrical signal that can be correlated to radiation dose. A MPPC also converts the light signal from the scintillation detector 103 into an electrical signal that can be correlated to radiation dose. The MPPC includes may small avalanche photodiodes that are sensitive to the scintillation light. The MPPC offers a lower cost and better durability than a PMT with a tradeoff in sensitivity. An array of multiple readout devices 212 allows for simultaneous realtime acquisition of signals from multiple scintillation detectors 103. For example, an array of five PMTs may be used in the detector interface 118 of FIG. 1 to allow simultaneous acquisition of data from the five scintillation detectors 103.

Data from each readout device 212 is routed through a routing hub 403 such as a serial-to-USB hub (e.g., UPort 1610-8, Moxa Inc., Brea, Calif.) via connections 406 such as, e.g., RS-232 cables and subsequently transferred from the routing hub 403 to a computing device 121 via a connection 409 such, e.g., as a USB cable. To limit the number of spurious pulses detected due to scattered x rays reaching the readout devices 212 and causing photocathode emissions, the housing of the detector interface 118 may be lined with lead shielding with a thickness of, e.g., about 1/16 inch. The detector interface 118 may also include a power supply for the readout devices 212 and the routing hub 403.

The computing device 121 may comprise, for example, a processor-based system such as a computer system. Such a computer system may be embodied in the form of, e.g., a desktop computer, a laptop computer, tablet computer systems, or other devices with like capability. The computing device 121 includes a display device upon which various screens, network pages, and other content may be rendered. The computing device 121 may be configured to execute various applications such as a field alignment application, a browser application, and/or other applications.

The example of FIG. 4 illustrates a FOC dosimeter 200 including an optical fiber 206 (FIG. 2B) connected to a first readout device 212a and a reference fiber 221 (FIG. 2B) connected to a second readout device 212b. As discussed above, measurements from the optical fiber 206 connected to a scintillator 203 (FIG. 2B) and from the reference fiber 221 may be obtained concurrently to compensate for the stem effect of optical fiber 206. As can be understood, FOC dosimeters 200 without a reference fiber 221 may also be utilized by connecting to a single readout device 212.

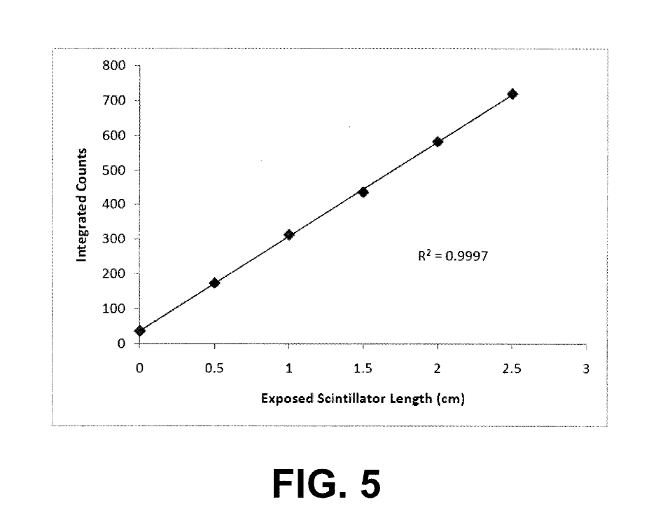

Referring back to FIG. 1, with the scintillation detectors 103a (e.g., FOC dosimeters 200) aligned optically along the edges of the light field 106, the scintillation detectors 103a scintillate during irradiation by the x-ray field 109. The counts are registered by the corresponding readout devices 212 (FIGS. 2A and 2B) during exposure. The total number of counts correlates to the length of the scintillation detector 103a exposed to the x-ray field 109 and thus a distance between the two fields. Hence, all sides of the field along with the beam intensity can be evaluated simultaneously. FIG. 5 is an example of a graph of integrated counts versus exposed scintillator length (cm) for an embodiment of the system 100 (FIG. 1) for digital x-ray field and light field alignment. Characterization included varying an exposed length of a 5 cm scintillator 203 of a FOC dosimeter 200 (FIGS. 2A and 2B) within the x-ray field. As can be seen in FIG. 5, the system response as a function of exposed length of the scintillator 203 is linear. By consistently placing the dosimeters in the same spot with respect to the light field, the deviation between the x-ray/light fields can be measured.

As illustrated in FIG. 1, additional scintillation detectors 103b may also be positioned within the center portion of the light field 106 to detect beam output. For example, a scintillation detector 103b may be substantially centered within the light field 106 to detect x-ray beam output level at that position. Other scintillation detectors 103b may be distributed within the light field 106 to characterize the beam output over the exposure window defined by the light field 106. The system 100 may also include other sensitive elements configured to collect a variety of other radiological quality assurance measurements that are distributed within the light field 106 and coupled to the detector interface 118. The sensitive elements may be configured to measure dosimetry or quality characterization information such as, e.g., half-value parameters. This information may be obtained concurrently with the field alignment information.

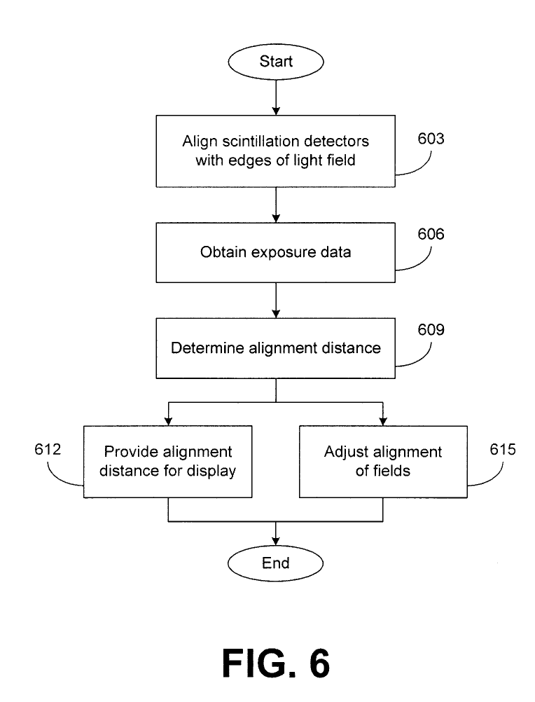

Referring now to FIG. 6, shown is a flow chart illustrating an example of field alignment using the system 100 of FIG. 1. Beginning with block 603, a plurality of scintillation detectors 103a are arranged about the edges of a light field 106. For example, a scintillation detector 103a may be positioned at about the center of each edge of the light field 106 as illustrated in FIG. 1. The scintillation detectors 103a may be positioned so that they are substantially perpendicular to the corresponding edge of the light field 106 and the scintillation detectors 103a may be centered on the light field edge to account for alignment variations between the light field 106 and the x-ray field 109 in either direction. In the example of FIG. 1, at least two scintillation detectors 103a are substantially perpendicular to each other and at least two scintillation detectors 103a are on opposite sides of the light field 106 and substantially in-line with each other. In some cases, the scintillation detectors 103a on opposite sides of the light field 106 may be offset but substantially parallel with each other.

In block 606, exposure data is obtained from the plurality of scintillation detectors 103a during irradiation by an x-ray field 109 associated with the light field 106. The exposure data includes the scintillation counts registered by a readout device 212 coupled to the scintillation detector 103a. The exposure data may also include the time corresponding to each registered count, the total number of counts registered during a predefined irradiation interval, the total number of counts from a reference fiber 221, or other information as can be appreciated. The exposure data may be simultaneously collected from the plurality of scintillation detectors 103a during the predefined irradiation interval. Other information may include, e.g., dosimetry or quality characterization information may also be obtained using other sensitive elements. The exposure data may then be provided to a computing device 121, e.g., through a routing hub 403.

In block 609, an alignment distance between the light field 106 and the x-ray field 109 is determined based at least in part upon the exposure data by the computing device 121. The total number of counts registered may be correlated to the displacement between the two fields 106 and 109 based upon, e.g., the linear relationship depicted in FIG. 5. For example, the distance may be determined based upon the exposed scintillator length and the positioning of the scintillator 203 of a FOC dosimeter 200. A direction corresponding to the alignment distance may also be determined. In some implementations, a plurality of alignment distances and corresponding directions may be determined.

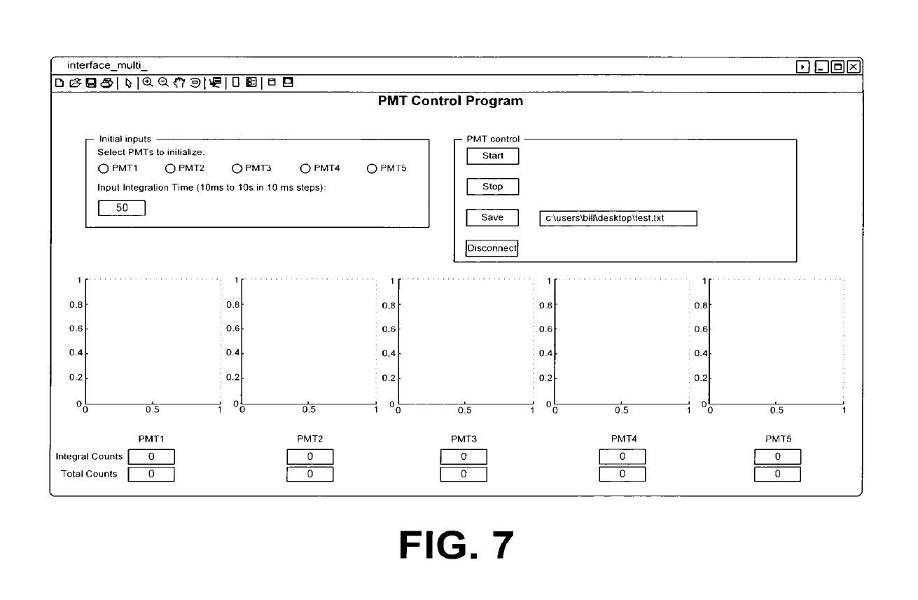

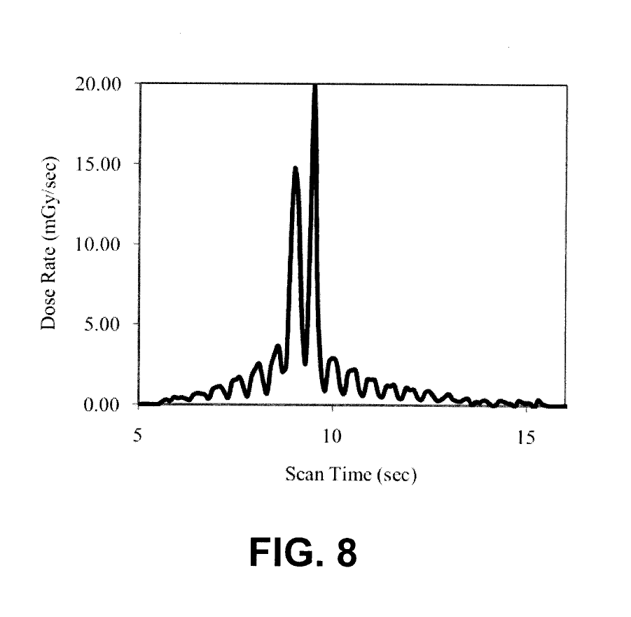

In block 612, the alignment information (distance and/or direction) may be provided for display by a display device. In some embodiments, a screen such as the one illustrated in FIG. 7 may be provided for display of realtime exposure data such as, e.g., integrated counts and counts with respect to time. FIG. 8 illustrates an example of realtime data collected with the system of FIG. 1 during a CT scan highlighting the ability of a FOC dosimeter to resolve quickly fluctuating dose rates. This permits simultaneous evaluation of the x-ray beam output profile, providing measures of beam constancy and exposure time. The screen may be interactive to provide for control of the field alignment determination through a field alignment application. The exposure information may be stored in memory associated with the computing device 121 for later access.

In block 615, the alignment information (distance and/or direction) may be used by the computing device 121 to adjust the alignment of x-ray field 109 with the light field 106. For example, the position of the x-ray field 109 may be adjusted based at least in part upon an alignment distance by reorienting the source of the x-ray field 109. The focus of the x-ray field 109 may be adjusted based at least in part upon a plurality of alignment distances. In other implementations, the light field 106 may be adjusted to correspond with the x-ray field 109. Control signals may be provided by the computing device 121 to control the adjustments of the x-ray field 109 and/or light field 106.

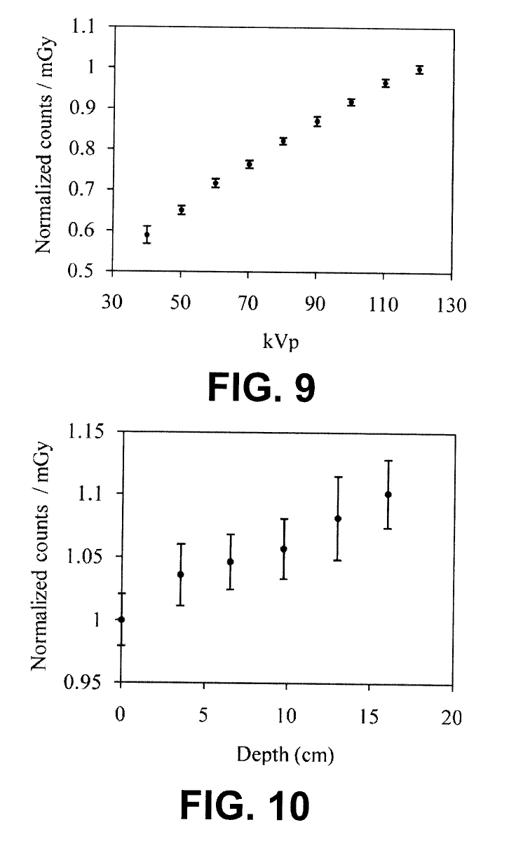

Other exposure data may also be obtained from scintillation detectors 103b within the light field 106 and used to determine the x-ray beam intensity and various other radiological measurements such as, e.g., the amount of radiation dose, radiation output rate, exposure time, and when combined with various attenuators x-ray beam energy and beam quality. FIG. 9 is an example of the energy dependence of a FOC dosimeter 200 in peak kilovoltage (kVp) applied across an x-ray tube of the source. The energy dependence of the FOC dosimeter 200 was evaluated by incrementally increasing the tube potential from 40 to 120 kVp in 10 kVp increments while maintaining the current-time product constant at 50 mA s. The data was normalized to the data point at 120 kVp. FIG. 10 is an example of the energy dependence of a FOC dosimeter 200 at 120 kVp as a function of depth in soft tissue-equivalent material. The data had been normalized to the surface measurement (i.e., depth=0). A plurality of scintillation detectors 103b distributed within the light field 106 may also be used to determine the x-ray distribution within the x-ray field 109.

The system disclosed herein permits rapid and accurate measurements of the x-ray/light field alignment for a mammography imaging system or other radiographic system. By determining the field misalignment using x-rays, this may be used to ensure the quality assurance (QA) of many types of radiographic systems. Additionally, the system has excellent reproducibility, a wide dynamic range of x-ray/light field deviation, and simultaneous data acquisition. The system includes minimal angular and energy dependence, insensitivity to common environmental variables, small size and light weight, and real-time output. Additionally, the small size and inert nature of the FOC dosimeters may make them useful for in-vivo dosimetry during imaging procedures. They may also exhibit a positive response to fast neutron and gamma ray irradiations.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A system, comprising:
a plurality of scintillation detectors distributed about edges of a light field associated with an x-ray field;
a detector interface in communication with the plurality of scintillation detectors, the detector interface configured to simultaneously obtain exposure data for the plurality of scintillation detectors; and
a computing device in communication with the detector interface, the computing device configured to determine an alignment distance between the light field and the x-ray field based at least in part upon the exposure data.

2. The system of claim 1, wherein the scintillation detectors are water-equivalent fiber optic coupled (FOC) dosimeters.

3. The system of claim 2, wherein the FOC dosimeters comprise a plastic scintillator coupled to an optical fiber.

4. The system of claim 3, wherein the FOC dosimeters further comprise a reference fiber.

5. The system of claim 2, wherein each of the scintillation detectors is substantially perpendicular to the corresponding edge of the light field.

6. The system of claim 5, wherein a scintillator included in each scintillation detector is centered about the corresponding edge of the light field.

7. The system of claim 5, wherein a first scintillation detector is substantially perpendicular to a second scintillation detector.

8. The system of claim 1, wherein the detector interface comprises a plurality of readout devices, each scintillation detector coupled to at least one of the readout devices.

9. The system of claim 8, wherein the detector interface further comprises a routing hub in communication with the plurality of readout devices, the routing hub configured to transfer exposure data from the plurality of readout devices to the computing device.

10. The system of claim 8, wherein the detector interface is lined with lead shielding.

11. The system of claim 1, further comprising a scintillation detector substantially centered within the light field.

12. A method, comprising:
obtaining exposure data from a plurality of scintillation detectors distributed about edges of a light field during irradiation by an x-ray field; and
determining an alignment distance between the light field and the x-ray field based at least in part upon the exposure data, the alignment distance corresponding to the distance between a first edge of the light field and a corresponding edge of the x-ray field.

13. The method of 12, further comprising determining a second alignment distance between the light field and the x-ray field based at least in part upon the exposure data, the second alignment distance substantially perpendicular to the first alignment distance.

14. The method of 12, wherein the exposure data includes a total number of counts registered during a predefined irradiation interval.

15. The method of 14, wherein the total number of counts is adjusted to account for stem effects.

16. The method of 12, wherein the exposure data includes a time corresponding to each registered count during the predefined irradiation interval.

17. The method of 12, further comprising providing the alignment distance for display on a display device.

18. The method of 17, further comprising providing an alignment direction corresponding to the alignment distance for display on the display device.

19. The method of 12, further comprising adjusting the x-ray field position based upon the alignment distance.

20. The method of 12, further comprising:
determining a plurality of alignment distances between the light field and the x-ray field based at least in part upon the exposure data; and
adjusting the focus of the x-ray field based at least in part upon the plurality of alignment distances.
